(19) United States
(12) Patent Application Publication
(10) Pub. No.: US 2015/0039239 A1
(43) Pub. Date: Feb. 5, 2015

(54) PROCESSOR-BASED METHODS, SYSTEMS AND PROGRAMS FOR REMOTE ANIMAL HEALTH MONITORING, ASSESSMENT, DIAGNOSIS, AND MANAGEMENT

(71) Applicant: AgLogica Holdings, LLC, Norcross, GA (US)

(72) Inventors: Richard Shuler, Johns Creek, GA (US); Marcel Sarzen, Atlanta, GA (US); Yongguo Hu, Suwanee, GA (US)

(21) Appl. No.: 14/446,908

(22) Filed: Jul. 30, 2014

Related U.S. Application Data
(60) Provisional application No. 61/860,512, filed on Jul. 31, 2013.

(51) Int. Cl. A61B 5/00

(52) U.S. Cl. CPC A61B 5/0015; A61B 5/48; A61B 5/72
USPC 702/19

(57) ABSTRACT

Processor-based methods and systems and computer programs for remote animal health monitoring receive and process data relating to animal health parameters obtained from a plurality of different types of sensors. Baseline data signatures are determined from the data obtained for individual animals, and as data is subsequently collected is compared to the current data signature to assess animal health. Deviations from the signatures may serve to predictively diagnose certain conditions and facilitate medical intervention before adverse physical symptoms are manifested. Informational data and analytics are made available to animal owners, health care providers, and other interested persons.

### CROSS REFERENCE TO RELATED APPLICATIONS

This application claims the benefit of U.S. Provisional Application Ser. No. 61/860,512 filed Jul. 31, 2013, the complete disclosure of which is hereby incorporated by reference in its entirety.

### BACKGROUND

This invention relates to generally to electronic systems and methods for remotely evaluating the health of animals, and still more specifically, to intelligent systems, methods and computer programs for real-time remote monitoring, evaluating, diagnosing and managing the health of a variety of different non-human animals with oversight and input by multiple human persons as well as via automatic sensed data collection.

Remote monitoring systems exist and are in use to assess animal health conditions. They are, however, problematic in some aspects and improvements are desired.

### BRIEF DESCRIPTION OF THE DRAWINGS

Non-limiting and non-exhaustive embodiments are described with reference to the following Figures, wherein like reference numerals refer to like parts throughout the various drawings unless otherwise specified.

FIG. 1 is a simplified block diagram of an exemplary animal health management system in accordance with one embodiment of the present invention.

FIG. 2 is an expanded block diagram of an exemplary embodiment of a server architecture of the animal health management system in accordance with one embodiment of the present invention.

FIG. 3 illustrates an exemplary configuration of a user system as shown in FIGS. 2 and 3.

FIG. 4 illustrates an exemplary configuration of a server system shown in FIGS. 2 and 3.

FIG. 5 is a process flow diagram of the exemplary animal health management system in accordance with one embodiment of the present invention.

FIG. 6 is a simplified schematic diagram of a portion of the exemplary animal health management system shown in FIG. 5.

FIG. 7 is a detailed schematic diagram of the exemplary animal health management system shown in FIG. 5.

FIG. 8 is more detailed process flow diagram of the exemplary animal health management system shown in FIG. 5.

### DETAILED DESCRIPTION OF THE DISCLOSURE

Exemplary embodiments of electronic, processor-based systems, methods and computer readable media for remotely monitoring, assessing, diagnosing, managing, and administrating the health care of animals are described below. As used in the following description, the term “health” shall include not only the conventional meaning, such as an animal being afflicted with a sickness or not, but also a behavior or a physiological state that animals may be experiencing, such as anxiety, estrus, or birthing. The inventive systems, methods and media address certain difficulties in the art, and in order to understand the invention to its fullest extent, set forth below is a discussion of the state of the art followed by description of exemplary concepts of the invention that overcome problems and difficulties in the art. Method aspects will be in part apparent and in part explicitly discussed in the disclosure below.

Unlike human persons, most other animals generally cannot communicate their overall condition or health status to a person who is capable of treating a health condition or ailment. This general inability to communicate includes but is not limited to communication of possible symptoms of a condition needing treatment that has not yet been diagnosed. Seeking timely medical care and treatment when necessary or advisable for such non-human animals therefore presents practical challenges that have yet to be fully resolved.

For example, a companion animal such as a dog, a cat or another household pet, if subjected to certain types of injury or illness, may not exhibit any physical symptoms for some time. The same is true of livestock animals such as cows, pigs or sheep for example. As such, when non-human animals become ill, their bodies are usually affected before any visual signs of the illness appear. Only when the symptoms are manifested in a way that humans can observe are the animals identified for possible treatment and diagnosis.

Veterinarians or other animal health care providers are able to check vital signs, as well as other factors, of a non-human animal to determine its health status. In the case of an animal in apparently good health (i.e., an animal that exhibits no apparent systems of illness), checkups by qualified, professional animal health care providers tends to be rather infrequent. In the case of an animal having an actual illness, however, checkups and evaluation tends to be well after the animal already contracted the medical condition and exhibits its effects. As noted above, because the animal lacks an ability to communicate with its owner or caretaker and because the animal's owner or caretaker is unable to detect any observable symptoms such conditions are highly unlikely to be appreciated at an earlier point in time wherein the condition less advanced and often may be more effectively treated.

Moreover, and adding further complications to the issues above, many animals, including human persons that can effectively communicate with other persons, often experience a period of time in which medical issues may exist without any physical symptoms being realized. In other words, animals may indeed be sick or in need of medical care for some time without consciously realizing it or without exhibiting symptoms that may be observed by others. In other words, an animal may actually have a health condition without subjective knowledge thereof, and also without any objective signs or symptoms that may be observed by others. While occasionally some medical conditions in this category are caught by chance, in most cases they are not. Preventative treatment and care could avoid or mitigate many health care issues of this type, but identifying such issues at early stages has proven elusive.

Remote monitoring systems are known that are designed to identify certain types of medical issues in non-human animals. Many of the monitoring systems in place suffer from the same problems noted above in that they detect health issues in non-human animals only when the animal exhibits observable symptoms. Early detection of a medical problem is very important in order to quickly assess and treat the problem to reduce animal suffering and to prevent further health and productivity complications which can develop if detection occurs late. Existing remote monitoring systems are generally disadvantaged in this regard as they tend to be designed to detect or identify certain specific characteristics in non-human animals that are often associated with specific symptoms of a specific condition, while ignoring other characteristics that may be indicators, positively or negatively, of animal health status.

In another aspect, apparently healthy non-human animals tend to be overlooked by existing remote monitoring systems, yet there is much value in assessing the health condition of these animals too. For example, accurate early detection of changing health conditions and events and changes in behavioral or physiological state in non-human animals depends, in part, on truly understanding and establishing so-called “normal” and “healthy” conditions. Existing health monitoring systems for non-human animals, however, are largely premised on assumptions regarding “normal” conditions of the animal, and again the systems are designed to identify symptoms of specific conditions that require treatment of affected animals, and perhaps isolation of affected animals to prevent transmission of certain conditions to other animals. The performance of these existing systems, of course, depends on the accuracy of the assumptions utilized in their operation. Known systems of this type lack a holistic approach to animal health care assessment in tracking and accounting for positive health conditions to more effectively evaluate negative ones.

Furthermore, in situations where there are many more animals than owners or caretakers such as a feed lot full of livestock or a zoo full of captive animals, time and budget constraints may make it very difficult or even prohibitive for an owner or caretaker to monitor the behavior and health status of each of the animals, whether individually or collectively. Each type of animal tends to present unique health care considerations and concerns, and addressing them in a comprehensive manner is needed. In particular, effective and simultaneous monitoring of different types of animals (e.g., cows, dogs and cats) with the same system presents practical challenges beyond the capability of known systems.

Accordingly, electronic, processor-based systems, methods and programs are needed that will allow owners of non-human animals, animal health care providers, and other interested persons, to remotely monitor the overall health of non-human animals, individually and collectively, in a more comprehensive and holistic manner to provide more effective early detection and diagnosis of changing health status, and perhaps even to predictively diagnose of animal conditions requiring intervention before physical symptoms are manifested.

Further, processor-based systems, methods and media are needed that allow a more complete and holistic assessment of healthy non-human animal conditions such that owners, animal health care providers and other interested persons can proactively promote, sustain, and perhaps even improve the condition of non-human animals in good health. By more comprehensively evaluating such healthy non-human animal conditions, interested persons can take proactive steps to optimize animal health such as, for example only, changing activity schedules, changing sleep schedules, adjusting animal diet and feeding times, introducing new activities and exercises for the animals, introducing nutritional supplements, and adjusting medicinal doses to minimize side effects. Such steps can likewise be adjusted as an animal grows, ages, and as its conditions and needs changes.

Such longstanding yet unfulfilled needs in the art are fulfilled by the inventive processor-based systems, methods and media described below. The following detailed description illustrates embodiments of the invention by way of example and not by way of limitation. The description clearly enables one skilled in the art to make and use the disclosure, describes several embodiments, adaptations, variations, alternatives, and uses of the disclosure, including what is presently believed to be the best mode of carrying out the disclosure. Exemplary computing device systems and methods of remotely monitoring the health of at least one non-human animal implemented with computing devices are disclosed wherein the animal's health is assessed by comparing a baseline health assessment for that animal to subsequently collected data and individualized behavioral/health state profiles.

It is contemplated that the inventive concepts disclosed have general application to computing systems in industrial, commercial, and residential applications insofar as monitoring of animal health is concerned. Further, while the invention is described in the context of monitoring and assessing health conditions of exemplary non-human animals, the invention is not necessarily limited to the exemplary animals described, and instead has a broader application to a variety of animals whether or not explicitly identified in the present disclosure, except as set forth in the attached claims. That is, the invention broadly accrues to monitoring of all types of animals.

Described herein are computer systems including computing devices. As described herein, all such computer systems include a processor and a memory. However, any processor in a computer device referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Wash.; and Sybase is a registered trademark of Sybase, Dublin, Calif.)

As used herein, the terms “software” and “firmware” are interchangeable, and include any computer program stored in memory for execution by processor 205, including RAM memory, ROM memory, EPROM memory, EEPROM memory, and non-volatile RAM (NVRAM) memory. The above memory types are exemplary only, and are thus not limiting as to the types of memory usable for storage of a computer program.

The technical effect of the processes and systems described herein is achieved when the system is provided with reference data such as that described below which, in turn may utilized in combination with sensed data collection and the exemplary algorithms and relationships described below to assess animal health states in a dynamic, real time manner that is believed to beyond the capability of known non-human animal health monitoring systems.

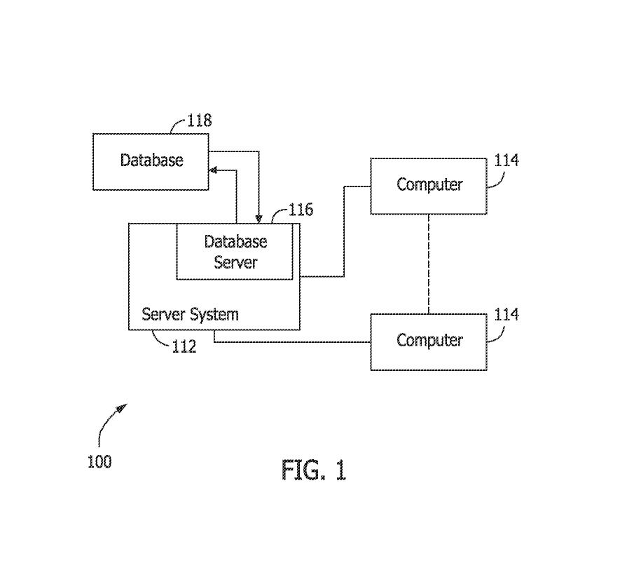

FIG. 1 is a simplified block diagram of an exemplary animal health monitoring (AHM) system 100 in accordance with one embodiment of the present invention. System 100 in the example shown is a cloud-based computing analysis system that receives data from multiple sources and performs analytics to assess the behavioral state of an individual animal by comparing a baseline data signature based on previously collected data to a current data signature based on data collected after the baseline data is established as described below.

More specifically, in the example embodiment, system 100 includes a server system 112, and a plurality of user sub-systems, also referred to as user systems 114, connected to server system 112. Computerized modeling and grouping tools, as described below in more detail, are stored in server system 112 and can be accessed by a requester at any one of user systems 114. In one embodiment, user systems 114 are computing devices such as computers or other electronic devices such as smartphones or tablets including a web browser, such that server system 112 is accessible to user systems 114 using, for example, the Internet.

User systems 114 may be interconnected to the Internet through many interfaces including, for example, a network such as a local area network (LAN) or a wide area network (WAN), dial-in-connections, cable modems, special high-speed ISDN lines, and inter-device transmission such as Bluetooth. User systems 114 may be or may include any computing device capable of interconnecting to the Internet including a web-based phone, personal digital assistant (PDA), or other web-based connectable equipment or equivalents thereof. A database server 116 is connected to a database 118 containing information on a variety of matters, as described below in greater detail. In one embodiment, database 118 is centralized and stored on server system 112, and database 118 be accessed by potential users at one of user systems 114 by logging onto server system 112 through one of user systems 114. In an alternative embodiment, database 118 is stored remotely from server system 112 and may be non-centralized.

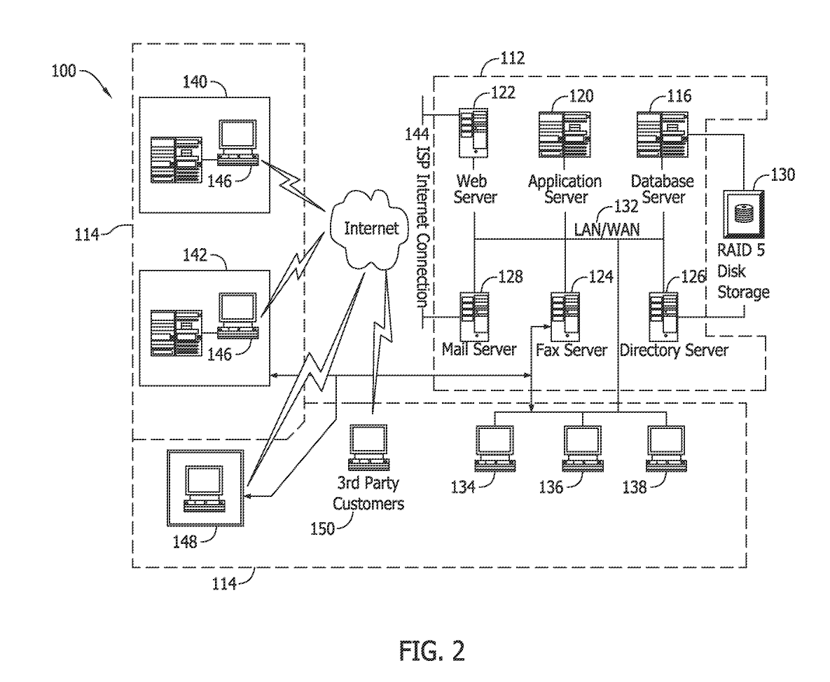

FIG. 2 is an expanded block diagram of an exemplary embodiment of a server architecture of AHM system 100 including server system 112 and user systems 114. Server system 112 includes the database server 116, an application server 120, a web server 122, a fax server 124, a directory server 126, and a mail server 128. A disk storage unit 130 is coupled to the database server 116 and the directory server 126. The servers 116, 120, 122, 124, 126, and 128 are coupled in a local area network (LAN) 132. In addition, a system administrator's workstation 134, a user workstation 136, and a supervisor's workstation 138 are coupled to the LAN 132. Alternatively, workstations 134, 136, and 138 are coupled to LAN 132 using an Internet link or are connected through an Intranet.

Each workstation, 134, 136, and 138 in contemplated embodiments may include a computing device such as a personal computer or other electronic device having a web browser. Although the functions performed at the workstations typically are illustrated as being performed at respective workstations 134, 136, and 138, such functions can be performed at one of many personal computers or other computing devices coupled to the LAN 132. Workstations 134, 136, and 138 are illustrated as being associated with separate functions only to facilitate an understanding of the different types of functions that can be performed by individuals having access to the LAN 132.

The server system 112 is configured or adapted to be communicatively coupled to various individuals via some of the user systems 114, including an animal owner or caretaker 140 associated with AHM system 100 that is responsible for the day-to-day care and well-being of the animal, and to an animal health care provider 142 such as a veterinarian that is responsible for diagnosing a medical condition of the animal, using, for example, an ISP Internet connection 144. The communication in the exemplary embodiment is illustrated as being performed using the Internet, however, any other wide area network (WAN) type communication can be utilized in other embodiments.

In an exemplary embodiment, any authorized individual having a workstation 146, 148 can access the server system 112 via one of user systems 114. At least one of user systems 114 includes a manager workstation 148 located at a remote location. Workstations 146 and 148 may be personal computers or other electronic computing devices having a web browser. Additionally, third party customers such as market research or clinical trial entities, may communicate with the server system 112 via a workstation 150 having, for example, a web browser.

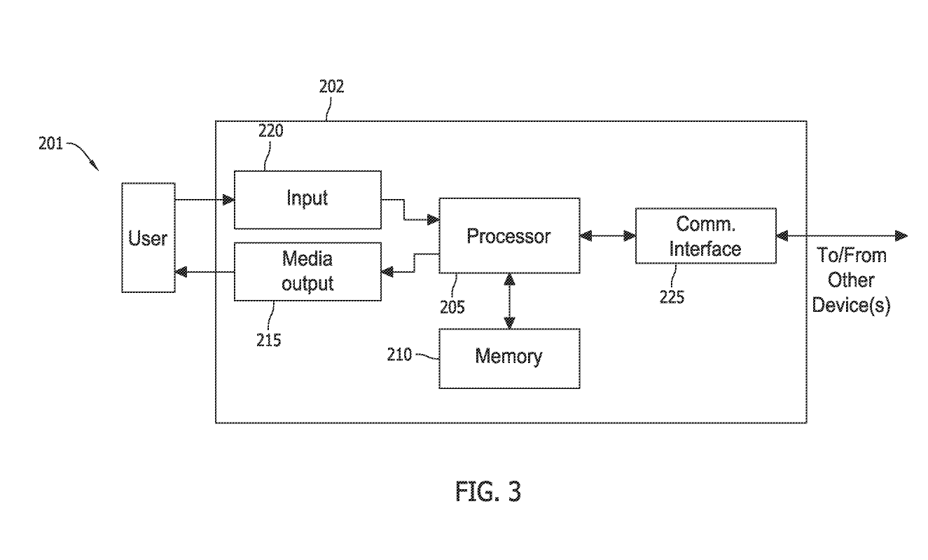

FIG. 3 illustrates an exemplary configuration of a computing device 202 that may be utilized to implement a user system 114 in the AHM system 100 of FIG. 2. More specifically, the computing device 202 may be utilized to implement the workstations 134, 136, 138 of the user systems 114 as well as the workstations 146, 148, and 150 in the AHM system 100 shown in FIG. 2. While a single computing device 202 is illustrated that could be used to implement any of the workstations 134, 136, 138, 146, 148, and 150 in the AHM system 100, different types and configurations of computing devices 202 could be used to implement the workstations 134, 136, 138, 146, 148, and 150 as desired.

In the example shown, computing device 202 includes a processor 205 for executing instructions stored in a memory 210. Processor 205 may include one or more processing units (e.g., in a multi-core configuration). Memory 210 may be or may include any device allowing information such as executable instructions and/or other data to be stored and retrieved. Memory 210 may include one or more computer readable media or programs to effect the data processing explained below. The computer readable media may be provided in the form of software having code segments effecting the data input, data collection, data processing, algorithmic analysis, and informational outputs described below.

The computing device 202 as shown includes at least one media output component 215 for presenting information to a user 201. The user 201 in contemplated embodiments is a person that is or may be associated with an animal owner, an animal health care provider or another interested person in animal health. Media output component 215 may be or may include any component capable of conveying information to user 201. In some embodiments, media output component 215 includes an output adapter such as a video adapter and/or an audio adapter. An output adapter is operatively coupled to processor 205 and operatively couplable to an output device such as a display device (e.g., a liquid crystal display (LCD), organic light emitting diode (OLED) display, cathode ray tube (CRT), “electronic ink” display or an audio output device (e.g., a speaker or headphone).

In some embodiments, the computing device 202 includes an input device 220 for receiving input from user 201. Input device 220 may include, for example, a keyboard, a pointing device, a mouse, a stylus, a touch sensitive panel (e.g., a touch pad or a touch screen), a gyroscope, an accelerometer, a position detector, a temperature sensor, a proximity sensor, or an audio input device in addition to other sensory devices. A single component such as a touch screen may function in some embodiments as both an output device of media output component 215 and input device 220.

The computing device 202 may also include a communication interface 225, which is communicatively couplable to a remote device such as server system 112. Communication interface 225 may include, for example, a wired or wireless network adapter or a wireless data transceiver for use with a communication network including but not limited to a mobile device network (e.g., Global System for Mobile communications (GSM), 3G, 4G or Bluetooth) or other mobile data network (e.g., Worldwide Interoperability for Microwave Access (WIMAX)).

Stored in memory 210 are, for example, computer readable instructions for providing a user interface to user 201 via media output component 215 and, optionally, receiving and processing input from input device 220. A user interface may include, among other possibilities, a web browser and client application. Web browsers enable users to display and interact with media and other information typically embedded on a web page or a website from server system 112. A user application allows user 201 to interact with a server application from server system 112.

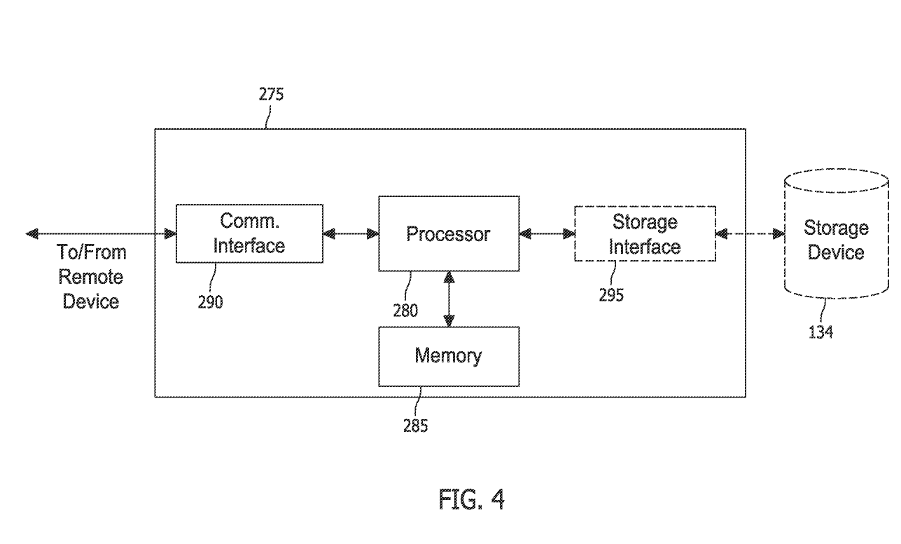

FIG. 4 illustrates an exemplary configuration of a server computing device 275 of server system 112 as shown in FIGS. 1 and 2. Server computing device 275 may include, but is not limited to, database server 116, transaction server 124, web server 126, fax server 128, directory server 130, and mail server 132 (shown in FIG. 2).

Server computing device 275 includes a processor 280 for executing instructions. Instructions may be stored in a memory 285, for example. Processor 280 may include one or more processing units (e.g., in a multi-core configuration).

Processor 280 is operatively coupled to a communication interface 290 such that server computing device 275 is capable of communicating with a remote device such as computing device 202 (FIG. 3) or another server computing device 275. For example, communication interface 290 may receive requests from client systems 114 via the Internet, as illustrated in FIGS. 1 and 2.

Processor 280 may also be operatively coupled to a storage device 134. Storage device 134 may be any computer-operated hardware suitable for storing and/or retrieving data. In some embodiments, storage device 134 is integrated in server computing device 275. For example, server computing device 275 may include one or more hard disk drives as storage device 134. In other embodiments, storage device 134 is external to server computing device 275 and may be accessed by a plurality of server computing devices 275. For example, storage device 134 may include multiple storage units such as hard disks or solid state disks in a redundant array of inexpensive disks (RAID) configuration. Storage device 134 may include a storage area network (SAN) and/or a network attached storage (NAS) system.

In some embodiments, processor 280 is operatively coupled to storage device 134 via a storage interface 295. Storage interface 295 may be any component capable of providing processor 280 with access to storage device 134. Storage interface 295 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 280 with access to storage device 134.

Memory 210 and 285 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

While the AHM system 100 described thus far is a cloud-based or server-based computer system, it is recognized that neither is required in other contemplated embodiments. The cloud-based or server-based system is beneficial when large numbers of animals are simultaneously monitored in disparate geographical regions and overseen by a large number of users with the system, but the AHM system can alternatively be implemented on a smaller scale with similar benefits.

For example, the AHM system may be configured to operate completely on computing devices such as a personal computer or notebook computer, or tablet computer as examples only. Personal or notebook computers, as well as other computing devices, may be interconnected with one another to provide certain functionality described below. For example, an animal owner may possess a personal computer or notebook computer having segments of code stored thereon allowing data to be processed and stored thereon, and an animal care provider may possess a personal computer or notebook computer also having segments of code stored thereon allowing data to be processed and stored thereon. Each of the animal owner and health care provider can individually access data on their respective computers, with the respective computers of the animal owner and health care provider sharing data with one another where needed. In some embodiments wherein the animal owner and health care provider are one and the same, a single computer may suffice to implement most, if not all, of the functionality described herein.

In another contemplated embodiment, segments of code corresponding to certain functionality as described herein can be downloaded or otherwise installed to a tablet computer or smartphone device, or other mobile or handheld processor-based device as an application to be enjoyed by an animal care owner or health care provider. Again, pertinent data may be transmitted from one user (e.g., an animal owner) to another user (e.g., an animal care provider) as desired or as needed using the mobile devices.

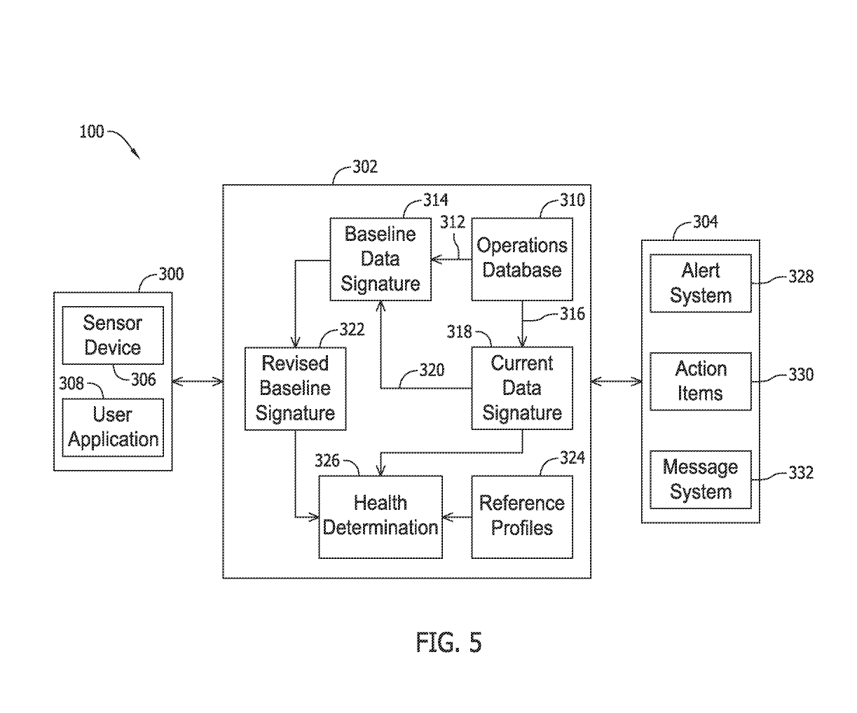

FIG. 5 is a process flow diagram of AHM system 100 configured to monitor, manage, and diagnose the health status of at least one animal that, among other things, is configured to predict whether or not an illness is oncoming before visible signs of the illness occur.

AHM system 100 in the example shown includes a data collection system 300, a data analysis system 302, and a user interface dashboard system 304. Alternatively, AHM system 100 may not include all three systems 300, 302, and 304, but may include only one or two of the systems 300, 302, and 304. For example, in various embodiments the system may include only the data collection system 300 for monitoring the health status of at least one animal, only the data analysis system 302 for managing the health of at least one animal, or only the user interface dashboard system 304 for diagnosing the health status of at least one animal, while still enabling the functionality described. As one example of this, the data can be collected and organized in a way that easily facilities the analytics explained below, but without actually performing the analytics. As another example, the system may perform analytics on data that is not itself collected by the system, and as such the data collection system 300 may be omitted. It should also be understood that functions of the data collection, analytic, and user interface systems 300, 302, and 304 could be combined into a single computing system or other numbers of computing systems than the three systems 300, 302, and 304 shown in FIG. 5, as well as distributed amongst other numbers of computing systems than the systems 300, 302, and 304 may be provided. While an exemplary architecture of the system is shown and described for discussion purposes, various different architectures are possible for the AHM system.

Furthermore, AHM system 100 may be flexible applied to assess animal health of various animals including companion animals such as dogs and cats as well as livestock animals such as cows and pigs. Embodiments of AHM system 100 may also be applied to both companion animals and livestock animals simultaneously. Additionally, AHM system 100 may be applied to monitor and assess the health of only a single animal, whether a companion or a livestock animal. The AHM system 100 may also be used with a population of animals such as multiple companion animals or a herd of livestock animals. Generally, AHM system 100 may be used with any number of a type of animal, and/or may be applied to monitor health of any number of different types of animals.

Data collection system 300 monitors the health status of at least one animal and includes a data collection sensor device 306 for collecting sensed data representing various health parameters of the animal and a user application 308 for collecting user observed data representing various behaviors of the animal. Data collection sensor device may associated with one or more of user systems 114 (FIG. 2) and may be a time sequenced quantitative sensor device that collects and stores a plurality of sensor data associated with behavioral traits and/or physiological conditions of a subject animal at predetermined time intervals over a predetermined time period, such as hourly intervals over a 24-hour period.

In the exemplary embodiment, data collection sensor device 306 includes a plurality of sensors of different types for measuring various behaviors and physiological conditions of the animal. Such sensors include, but are not limited to, an accelerometer to measure the animal's movement, a GPS sensor to track the animal's location, a first thermometer for measuring the ambient air temperature around the animal, a second thermometer for measuring the body temperature of the animal, a pedometer for registering the amount of steps taken by the animal, a microphone for registering any noises produced by the animal, a camera for viewing the animal's surrounding environment, and various physiological sensors for measuring the animal's heart rate, blood pressure, breathing rate, food/water intake and urination/defecation events. AHM system 100 processes the sensed data as explained below to assess changes in the well-being, medical status, and behavior of the animal.

In the exemplary embodiment, data collection sensor device 306 is coupled to a collar worn by the animal. The collar includes a ruggedized housing that is water and shock resistant such that data collection sensor device 306 is protected from external environmental hazards. Furthermore, data collection sensor device 306 may include a rechargeable, low-voltage energy source and a battery indicator means to indicate the remaining battery life of device 306 before it must be re-charged.

Data collection sensor device 306 further includes a data transmission means such as transmitter or transceiver for transmitting data to data analysis system 302. In contemplated embodiments, the data collection sensor device 306 may include a wireless transceiver having a range of 30 to 60 meters, which is suitable for most domestic applications. In a farm environment, the transceiver may have a range of about 1 mile or more. The data gathered from the animal is stored in a non-volatile memory unit on sensor device 306. At predetermined or intermittent times, data collection sensor device 306 sends time and date stamped sensed data by means of a data transmission protocol to data analysis system 302. The data transmission protocol can be chosen from many different systems known in the art, including, but not limited to, wireless LAN such as Wi-Fi, or machine-to-machine transmission such as Bluetooth. In the exemplary embodiment, a periodic data transmission is used in order to conserve the battery charge of data collection sensor device 306 and extend its use before having to be re-charged or replaced.

Data transmission may also occur from the data collection sensor device 306 when the animal comes within range of a base station that relays the sensed data from device 306 to system 302. If data collection sensor device 306 is not within range of the base station, data collection sensor device 306 stores the sensed data in its onboard memory. When the animal returns to a location within the predetermined range of the base station, data collection sensor device 306 transmits the sensed data to data analysis system 302. In contemplated embodiments, data collection sensor device 306 is able to store a large amount of data such that when data transmission may not occur for some time complete data sets are nonetheless collected. The data collection sensor 306 may optionally begin to overwrite the oldest data once the memory is full, although this will undesirably result in gaps in the data collected and present related data processing issues.

User application 308 may be provided in one or more of user systems 114 as illustrated in FIG. 2, and may be a time sequenced qualitative application that allows an animal owner or caretaker to input individual behavior events or any other witnessed observations into data collection system 300. In an exemplary embodiment, user application 308 is a mobile device application for use with a mobile web-enabled computing device such as a smartphone or tablet. User application 308 enables an animal owner or caretaker to record time-stamped observed behavior events such as but not limited to changes in eating/drinking habits, changes in animal activity levels, scratching, vomiting, bowel events, and responses to various health treatments such as physical therapy or medication. As described above with reference to user system 112, user application 308 is communicatively coupled to data analysis system 302 for transmission of observed data to server system 112.

Data analysis system 302 may be a cloud-based system that manages the health of at least one animal by storing the data received from data collection sensor device 306 and user application 308 in an operations database 310 and performing analytics on the data to assess the behavior state of an individual animal using changes in the animal's health, nutrition, and/or physiological state. In the exemplary embodiment, data analysis system 302 is server system 112 described above. Data from data collection system 300 is analyzed by data analysis system 302 to generate an analytic data signature representing a combination of the received qualitative data from user application 308 and the quantitative data from data collection sensor device 306. Initially, the collected data from system 300 is stored in operations database 310 and is calibrated to determine various behavioral events of the animal. For example, sensed activities such as such as the number of steps taken by the animal, the animal's sleeping patterns, eating/drinking events, scratching events, etc. may be used to deduce an event such as the animal's anxiety level and determine whether or not an anxiety event is problematic.

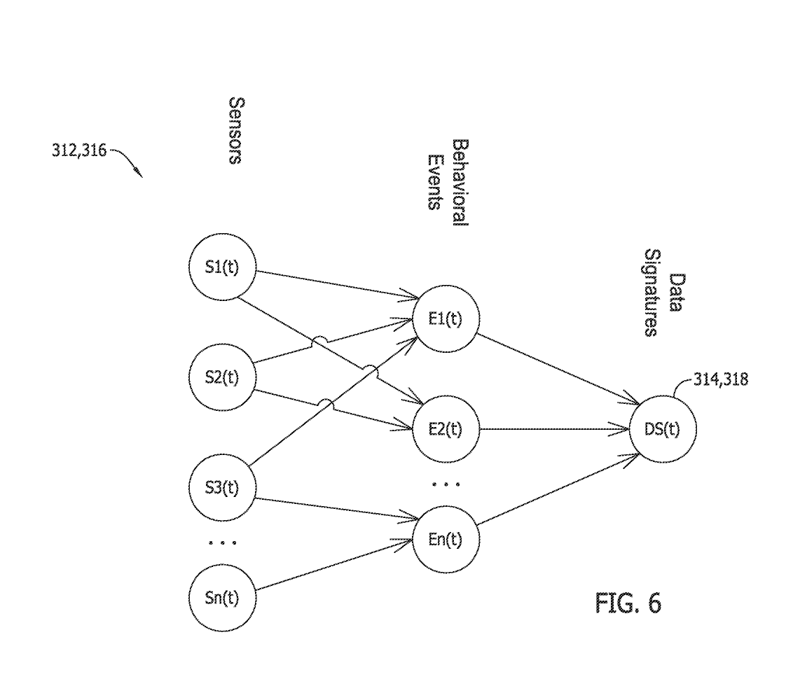

Various algorithms, represented here by arrow 312 and described in further detail in reference to FIG. 6, are applied to the collected data to determine a baseline data signature 314 that represents a starting behavioral/health state of an individual animal being monitored. The baseline signature 314 may be represented by a data table profile that may subsequently be used to indicate whether the animal is in pain, the animal is anxious, or the animal is in good health. Additionally or alternatively, algorithms 312, 314 may output a baseline health score that represents the overall baseline health condition of the individual animal being monitored. A baseline health score within different predetermined ranges may indicate different behavioral or health states of the animal. Unlike known systems, baseline data signature 314 is determined based on the data collected from a plurality of sensors in data collection sensor device 306 and from recorded observations made by a user in user application 308 such that baseline data signature 314 is based on an aggregate of data from all the collected data and not a single selected sensor type. For example, baseline signature 314 may be based on movement data from an accelerometer, location data from a GPS unit, temperature data from at least one thermometer, audio data from a microphone, and heart rate, blood pressure, breathing rate from various physiological sensors.

After baseline data signature 314 has been determined, data analysis system 302 is configured to receive data from data collection system 300 that is collected after the data used to establish baseline signature 314. A current data signature 318 is generated using algorithms represented here by arrow 316, and described in further detail below with reference to FIG. 6, that represents the most recent behavioral and/or health state of the animal. For example, current signature 318 may be represented by a data table profile that may indicate that the animal is in pain, the animal is anxious, or the animal is in good health. Additionally or alternatively, algorithms 316 may represent current data signature 314 as a current health score that represents the overall current health condition of the individual animal being monitored. A current health score within different predetermined ranges may indicate different behavioral or health states of the animal depending on the range. Baseline data signature 314 is updated over time with subsequently collected data via a feedback loop 320 to reflect changes in the behavioral state of the animal such as responses to health treatments. The incorporation of current data signature 318 with baseline data signature 314 via feedback loop 320 generates a revised baseline signature 322 that is used to assess the health of the animal as described below. Similar to baseline signature 314 and unlike known systems, current data signature 318 is determined based on the data collected from a plurality of sensors in data collection sensor device 306 and from recorded observations made by a user in user application 308 such that current data signature 318 is based on an aggregate of data from all the collected data and not a selected sensor type. For example, current signature 318 may be based on movement data from an accelerometer, location data from a GPS unit, temperature data from at least one thermometer, audio data from a microphone, and heart rate, blood pressure, breathing rate from various physiological sensors.

Data analysis system 302 also includes a reference health and physiological state profiles database 324 that includes health, nutrition, and physiological state profiles that represent thresholds of change for indicating a significance in change between baseline signature 314 and current signature 318 for the individual animal. In the exemplary embodiment, reference health and physiological state profiles database 324 includes health, nutrition, and physiological state profiles that are based on previously collected data that is stored on database 310 for the individual animal and may be utilized to indicate significant changes between baseline data signature 314 and current data signature 318. Alternatively, the reference health state profiles may be based on data collected from an animal that is not the subject animal being monitored, but is of the same species.

Similar to baseline signature 314 and current signature 318, the health, nutrition, and physiological state profiles may be represented either as a table of collected data over a predetermined period of time or by a predetermined range of health score. Alternatively, the reference health state profiles may be represented by weighing factors that are applied to baseline and current data signatures 314 and 316. In an exemplary embodiment, reference health and physiological state profiles database 324 includes health, nutrition, and physiological state profiles that indicate at least one of the following health, physiological, or behavioral states: healthy, pain, estrus, rumination, reduced mobility, birthing, anxiety, ear infection, medication side effects, body weight fluctuation, bodily function events, and food/water intake events. A number of exemplary health state reference profiles are provided below in Tables 1-7, wherein each row represents the data sensed by a different sensor and each column represents the number of events registered by each individual sensor for a specific hour of the day:

TABLE 1
Healthy Profile

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 50 | 90 | 90 | 90 | 90 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 1 | 1 | 1 | 1 | 0 | 1 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 1 | 0 | 1 |
| Water | 0 | 0 | 0 | 0 | 0 | 0 | 2 | 0 | 1 | 0 | 0 | 1 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 90 | 90 | 90 | 90 | 90 | 400 | 80 | 40 | 0 | 0 | 0 | 0 |
| GPS | 5 | 5 | 5 | 5 | 5 | 100 | 5 | 5 | 0 | 0 | 0 | 0 |
| Accelerometer | 0 | 2 | 1 | 1 | 1 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 1 | 0 | 0 | 1 | 0 | 0 | 0 | 0 | 0 | 0 |
| Feed | 0 | 0 | 0 | 0 | 0 | 1 | 0 | 0 | 0 | 0 | 0 | 0 |

TABLE 2
Anxiety Profile

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 100 | 175 | 200 | 225 | 200 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 9 | 15 | 20 | 5 | 30 | 15 |
| Temp | 72 | 72 | 72 | 72 | 72 | 72 | 72 | 72 | 72 | 72 | 72 | 72 |
| Audiometer | 0 | 0 | 0 | 0 | 0 | 0 | 2 | 4 | 1 | 2 | 2 | 1 |
| Water | 0 | 0 | 0 | 0 | 0 | 0 | 2 | 0 | 1 | 0 | 0 | 1 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 175 | 150 | 150 | 90 | 90 | 400 | 80 | 40 | 0 | 0 | 0 | 0 |
| GPS | 5 | 5 | 5 | 5 | 5 | 100 | 5 | 5 | 0 | 0 | 0 | 0 |
| Accelerometer | 21 | 9 | 9 | 9 | 0 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 5 | 2 | 0 | 0 | 0 | 0 | 0 | 0 | 0 | 0 | 0 |
| Water | 0 | 0 | 1 | 0 | 0 | 1 | 0 | 0 | 0 | 0 | 0 | 0 |
| Feed | 0 | 0 | 0 | 0 | 0 | 1 | 0 | 0 | 0 | 0 | 0 | 0 |

TABLE 3
Ear Infection Profile

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 50 | 90 | 90 | 90 | 90 |
| GPS | 0 | 0 | 0 | 0 | 0 | 0 | 0 | 20 | 5 | 5 | 5 | 5 |
| Accelerometer | 5 | 8 | 6 | 8 | 9 | 9 | 5 | 8 | 6 | 8 | 9 | 9 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 1 | 0 | 1 |
| Water | 0 | 0 | 0 | 0 | 0 | 0 | 2 | 0 | 1 | 0 | 0 | 1 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 90 | 90 | 90 | 90 | 90 | 400 | 80 | 40 | 0 | 0 | 0 | 0 |
| GPS | 5 | 5 | 5 | 5 | 5 | 100 | 5 | 5 | 0 | 0 | 0 | 0 |
| Accelerometer | 5 | 8 | 8 | 5 | 8 | 6 | 8 | 9 | 9 | 5 | 8 | 8 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 1 | 0 | 0 | 1 | 0 | 0 | 0 | 0 | 0 | 0 |
| Feed | 0 | 0 | 0 | 0 | 0 | 1 | 0 | 0 | 0 | 0 | 0 | 0 |

TABLE 4
Pain Profile 1

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 5 | 10 | 5 | 10 | 5 | 10 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 1 | 1 | 1 | 1 | 0 | 1 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 1 | 0 | 1 |
| Water | 0 | 0 | 0 | 0 | 0 | 0 | 2 | 0 | 1 | 0 | 0 | 1 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 5 | 10 | 5 | 10 | 5 | 10 | 5 | 10 | 0 | 0 | 0 | 0 |
| GPS | 5 | 5 | 5 | 5 | 5 | 100 | 5 | 5 | 0 | 0 | 0 | 0 |
| Accelerometer | 0 | 2 | 1 | 1 | 1 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 1 | 0 | 0 | 1 | 0 | 0 | 0 | 0 | 0 | 0 |
| Feed | 0 | 0 | 0 | 0 | 0 | 1 | 0 | 0 | 0 | 0 | 0 | 0 |

TABLE 5
Pain Profile 2

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 50 | 90 | 90 | 90 | 90 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 0 | 0 | 0 | 0 | 1 | 0 | 0 | 0 | 0 | 0 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 90 | 90 | 90 | 90 | 90 | 400 | 80 | 40 | 0 | 0 | 0 | 0 |
| GPS | 5 | 5 | 5 | 5 | 5 | 100 | 5 | 5 | 0 | 0 | 0 | 0 |
| Accelerometer | 0 | 0 | 0 | 0 | 0 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 0 | 0 | 0 | 1 | 0 | 0 | 0 | 0 | 0 | 0 |
| Feed | 0 | 0 | 0 | 0 | 0 | 1 | 0 | 0 | 0 | 0 | 0 | 0 |

TABLE 6
Food/Water Intake Profile

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 50 | 90 | 90 | 90 | 90 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 1 | 1 | 1 | 1 | 0 | 1 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 1 | 0 | 1 |
| Water | 0 | 0 | 0 | 0 | 0 | 0 | 1 | 0 | 0 | 0 | 0 | 0 |
| Feed | 0 | 0 | 0 | 0 | 0 | 0 | 1 | 0 | 0 | 0 | 0 | 0 |
| Hour | 13 | 14 | 15 | 16 | 17 | 18 | 19 | 20 | 21 | 22 | 23 | 24 |
| Pedometer | 90 | 90 | 90 | 90 | 90 | 400 | 80 | 40 | 0 | 0 | 0 | 0 |
| GPS | 5 | 5 | 5 | 5 | 5 | 100 | 5 | 5 | 0 | 0 | 0 | 0 |
| Accelerometer | 0 | 2 | 1 | 1 | 1 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 0 | 0 | 0 | 0 | 0 |
| Water | 0 | 0 | 0 | 0 | 0 | 1 | 0 | 0 | 0 | 0 | 0 | 0 |
| Feed | 0 | 0 | 0 | 0 | 0 | 0 | 0 | 0 | 0 | 0 | 0 | 0 |

TABLE 7
Medication Impact Profile

| Hour | 1 | 2 | 3 | 4 | 5 | 6 | 7 | 8 | 9 | 10 | 11 | 12 |
|---|---|---|---|---|---|---|---|---|---|---|---|---|
| Pedometer | 0 | 0 | 0 | 0 | 0 | 0 | 25 | 75 | 120 | 150 | 120 | 95 |
| GPS | 0 | 0 | 0 | 0 | 0 | 0 | 0 | 20 | 5 | 5 | 5 | 5 |
| Accelerometer | 0 | 0 | 0 | 0 | 0 | 0 | 4 | 3 | 5 | 6 | 8 | 4 |
| Temp | 72 | 72 | 72 | 72 | 72 | 72 | 72 | 72 | 72 | 72 | 72 | 72 |
| Audiometer | 0 | 0 | 0 | 0 | 0 | 0 | 0 | 3 | 0 | 4 | 5 | 7 |
| Water | 0 | 0 | 0 | 0 | 0 | 2 | 2 | 1 | 2 | 2 | 0 | 3 |
| Feed | 0 | 0 | 0 | 0 | 0 | 1 | 1 | 0 | 0 | 0 | 0 | 0 |
| Hour | 13 | 14 | 15 | 16 | 17 | 18 | 19 | 20 | 21 | 22 | 23 | 24 |
| Pedometer | 130 | 120 | 90 | 75 | 150 | 400 | 90 | 300 | 0 | 0 | 0 | 0 |
| GPS | 5 | 5 | 5 | 5 | 5 | 100 | 5 | 5 | 0 | 0 | 0 | 0 |
| Accelerometer | 3 | 2 | 4 | 7 | 3 | 0 | 0 | 0 | 0 | 0 | 0 | 0 |
| Temp | 72 | 72 | 72 | 72 | 72 | 72 | 72 | 72 | 72 | 72 | 72 | 72 |
| Audiometer | 4 | 3 | 0 | 4 | 5 | 0 | 0 | 0 | 0 | 0 | 0 | 0 |
| Water | 0 | 2 | 3 | 0 | 0 | 2 | 0 | 2 | 0 | 0 | 0 | 0 |
| Feed | 1 | 0 | 0 | 0 | 0 | 1 | 0 | 0 | 0 | 0 | 0 | 0 |

In an exemplary embodiment, data analysis system 302 applies algorithm 312 or 316 to one of baseline signature 314 or revised baseline signature 322, current signature 318, and the profiles from reference health and physiological state profiles database 324 to assess and indicate significant changes in the animal's behavioral, physiological, or health state. Data analysis system 302 uses baseline signature 314 only when baseline signature 314 has not been revised by feedback loop 320. Data analysis system 302 uses algorithms 312 and 316 to compare one of baseline signature 314 or revised baseline signature 322 with current signature 318 and the reference health state profiles from reference health and physiological state profiles database 324 to generate a health assessment 326. In the exemplary embodiment, data analysis system 302 uses the health state reference profiles and collected data from the individual animal applied with weighing factors to assess the animal's health assessment 326 with respect to a particular one or more of the reference health state profiles provided. Changes in the animal's behavioral and/or health state reflected in the health assessment 326 may be indicated by displaying a visual representation such as but not limited to a chart or a graph. Alternatively, health assessment 326 may be indicated by a table of collected data or a health score.

When baseline signature 314 or 322, current signature 318, and reference health and physiological state profiles 324 are represented as a table of collected data, then algorithms 312 and 316 may facilitate representing health assessment 326 as a table of collected data. Similarly, when baseline signature 314 or 322, current signature 318, and reference health and physiological state profiles 324 are represented as a health score, then algorithms 312 and 316 also facilitate representing health assessment 326 as a health score. Data analysis system 302 may further use health assessment 326 to diagnose the animal with a health or behavioral condition needing treatment or intervention or otherwise deem the animal to be in good health based on either the table of collected data or on the health score. In the exemplary embodiment, the health assessment 326 may serve as a predictive indicator of whether or not an illness is oncoming based on baseline signature 314 or 322, current signature 318, and reference health and physiological state profiles 324 before visible signs of the illness occur. Furthermore, health assessment 326 may be used to monitor the animal's behavioral actions due to the implementation of a certain health treatment and/or dietary changes. That is, the behavioral and health effects of various health care treatments and lifestyle changes may be detected and assessed by the system.

FIG. 6 illustrates a schematic diagram of algorithms 312 and 316 (FIG. 5) that process data collected with a number n of different sensors S1(t), S2(t), S3(t), . . . Sn(t) via the data collection sensor device 306 (FIG. 5) in contemplated embodiments, a number n of different behavioral events E1(t), E2(t), . . . En(t) deduced from the collected data as described below, and one of baseline date signature 314 or current data signature 316. In an exemplary embodiment, each sensor S1(t), S2(t), S3(t), . . . Sn(t) respectively collects data related to that particular sensor at predetermined time intervals over a predetermined time period. For example, S1(t) may be an accelerometer that measures the number of times the animal raises and lowers its head during separate one hour time intervals over the course of a full 24-hour day. Further, S2(t) may be a microphone that measures the number of times certain sounds are produced during the same one hour intervals as the accelerometer is measuring head movement, and sensor S3(t) may be a GPS locator that represents the location of the animal during the same one hour intervals. The data collected by sensors S1(t), S2(t), and S3(t) during a predetermined time range may then be combined to identify at least one behavioral event, such as E1(t), that occurred during that predetermined time range.

For example, if sensor S1(t) collects data that represents multiple head lowering activities during a certain hour-long interval, sensor S2(t) collects data that represents sound was produced during the same hour-long interval, and sensor S3(t) collects data that represents that the animal was at the same location as its water dish during the same certain hour-long interval, then the behavioral event E1(t) determined by the combined data of sensors S1(t), S2(t), and S3(t) may be that the animal was drinking. However if sensors S1(t), S2(t), and S3(t) collect data that represents the head-lowering, sound production, and that the animal being near the water dish each occurred during different hour-long intervals instead of in the same interval, then the animal likely did not get a drink during any of those intervals and behavioral event E1(t) did not occur during those intervals. As shown in FIG. 6, each sensor of the plurality of sensors S1(t), S2(t), S3(t), . . . Sn(t) may be used in combination with other sensors within the plurality of sensors S1(t), S2(t), S3(t), . . . Sn(t) to identify behavioral events E1(t), E2(t), . . . En(t) that occurred within a predetermined time period. Any number n of sensors may be used to detect any number n of behavioral events that can be used to assess animal health with varying degrees of sophistication of the system.

Some behavioral events E1(t), E2(t), . . . En(t) may be detected by a single sensor in certain embodiments although this may introduce some ambiguity in determining the animal's actual condition. For example, a position sensor may indicate that the animal is moving and movement may be deemed a behavioral event for analysis by the system. The position sensor data, however, may not indicate whether the animal is walking, running, or being carried by a person or a moving vehicle. Feedback from other sensors, however, in combination with the position sensor may resolve such ambiguity. In this example, an accelerometer, a heart rate sensor, a microphone and/or a camera including in the data collection sensor device 306 may reliably reveal, in combination with the data from the position sensor, whether the animal is walking, running, or being carried. Walking, running and being carried could accordingly be behavioral events that are detected by the system. Because of possible ambiguities associated with single sensor events, a plurality of sensors are preferably used to detect behavioral events in contemplated embodiments. Utilizing a plurality of sensors also beneficially provides a degree of redundancy to the system. In the example above, the system may successfully detect whether the animal is walking, running or being carried even if one of the position sensor, accelerometer, heart rate sensor, microphone or a camera malfunctions and associated data for the malfunctioning sensor is not collected.

Once a number of behavioral events that occurred within the predetermined time period are identified, they may be input into algorithm 312 or 316, as described below, which determines a data signature 314 and/or 318 for the animal over the predetermined time period. Baseline data signature 314 is calculated with this method using the initial data collected by data collection sensor device 306 at the beginning of system 100 utilization. Similarly, current data signature 318 is determined with this method using subsequently collected data from data collection sensor device 306. As described below, differences between baseline data signature 314 and current data signature 318 are used to determine a wellness or behavioral change of the animal based on reference health and physiological state profiles 324.

In an exemplary embodiment, algorithm 312 is substantially similar to algorithm 316, and is used to determine baseline and current data signatures 314 and 318 after behavioral events E1(t), E2(t), . . . En(t) have been determined from the collected sensor data from device 306. An exemplary algorithm 312 is set forth below:

DS={[w₁E₁+w₂E₂+W₃E₃+ . . . +wₙEₙ]ₜ,tεT} (Algorithm 312)

where DS is one of data signatures 314 or 318, Eₙ are the behavioral event scores for the behavioral events selected based on the health state being monitored and/or assessed, t is time, and wₙ are the weighing factors that are based on the reference health and physiological state profiles 324 described above. More specifically, Eₙ is a behavioral state score that represents the sum of variances of each predetermined time interval of a certain activity (i.e., behavioral events) over the predetermined time period.

As used herein, the term “variance” is meant to be the difference in the number of activities detected in different time intervals. For example, the variance may be an activity registered by one of sensors S1(t), S2(t), or S3(t) during a certain predetermined time interval of a first day and the number of activities registered by the same sensor S1(t), S2(t), or S3(t) during the same time interval of a different day. This concept may also be termed “time-boxing.” For example, if sensor S1(t) is a pedometer that registers 100 steps taken by an animal between 9 am and 10 am on the day baseline data is collected, and sensor S1(t) registers 200 steps by the same animal during the same time interval of 9 am to 10 am on another day that current data is collected, then the variance between the baseline data and the current data in this interval is 100 steps. In an exemplary embodiment, the variance is the absolute value difference between the baseline data and the current data. Alternatively, the variance may include positive and/or negative values that may represent progress or regress of the animal's well-being with respect to specific health parameters.

The behavioral event score Eₙ used in algorithm 312 is the sum of the variances of each hour of a behavioral event over a predetermined time period, which is 24 hours in the exemplary embodiment. For example, if the variance of registered steps between baseline data and current data between 9 am and 10 am is 100 and if the variance of registered steps between baseline data and current data between 10 am and 11 am is 85, then the behavioral event score Eₙ for the hours between 9 am and 11 am is the sum of the variances (100 plus 85) or 185 steps. However, in order for the behavioral event score Eₙ to be used to determine data signatures 314 and 318, the behavioral event score Eₙ is weighed according to a certain reference health state or physiological state profile 324.

Predetermined weighing factors wₙ are applied to the summed variances of the behavioral event scores Eₙ in the algorithm 312 to weight the importance of a variation between the number of events in the baseline data signature and the current data signature. Weighing factors wₙ may be determined by reference to the health and physiological state profiles 324, wherein the different behavioral events of each health reference profile 324 are weighted differently according to which behavioral events best exemplify that profile 324. Additionally, a negative weighing factor wₙ may be applied to a behavioral event whose occurrence may indicate that a certain health or physiological state profile 324 does not fit the behavior of the animal as determined from the data collected. For example, in the anxiety profile of Table 2 above, a relatively high number of registered steps from the pedometer is highly indicative of an increased anxiety level in the animal, so a relatively high weight wₙ is given to pedometer readings in the anxiety profile. On the other hand, drinking events are much less relevant to determining the anxiety level of an animal, so the number of drinking events is given a low weight or perhaps even zero weight wₙ with respect to determining the animal's anxiety level with the algorithm 312. Furthermore, a barking event registered by the microphone may be an indicator of anxiety, but barking is not as correlated to anxiety as a high pedometer reading, so barking behavioral events may receive an intermediate weighing factor wₙ with respect to determining the animal's anxiety level. The behavioral event scores Eₙ from the collected data are weighed according to each of the reference health and physiological state profiles 324 such that a different data signature 314 or 318 may be generated for each profile 324. For example, in an embodiment including eight health reference profiles such as those shown in Tables 1-8 above, eight data signatures may be may be generated to assess an animal's health with respect to each of the eight health reference profiles. The respective data signatures in this example may each be calculated using the same baseline data and current data having different weighing factors wₙ applied to assess the animal with respect to each health reference profile. Alternatively, data signatures 314 and 318 may be calculated using weighing factors wₙ for only selected profiles 324 and not all profiles 324. That is, even when eight health reference profiles are provided, they need not all be used all of the time, and in some embodiments a user may select which of the health assessment profiles is to be utilized.

In an exemplary embodiment, algorithms 312 and 316 generate baseline and current data signatures 314 and 318, respectively, as a time based data series. Once baseline and current data signatures 314 and 318 are determined, health assessment 326 may be made utilizing the following exemplary comparative relationship:

If (DS_{current})>N*SDev(DS_{baseline}) then a significant behavioral change is flagged

where DS_{current} is the current data signature 318, DS_{baseline} is the baseline data signature 314, SDev is the standard deviation of the data series representing baseline data signature 314, and N is a multiple of standard deviations required to flag current data signature 318 based on the health or physiological state profile 324 being referenced. In contemplated embodiments N may range in value from 1 to 2, and more specifically N may range from 1.5 to 2, although in other embodiments other ranges defined by higher and lower values may alternatively be utilized instead.

In the example provided, the comparative relationship to determine the health assessment is in the form of an inequality. Alternatively, a significant behavioral change between baseline data signature 314 and current data signature 318 may be indicated by any relationship that allows system 100 to function as described herein. The standard deviation SDev and its multiple N are based on the reference health profiles 324 and represent a threshold factor that, when multiplied by the baseline data signature 314, signals a divergence from the baseline health or behavior condition (as reflected in the baseline data signature) that, it turn, triggers the system to provide a notification or alert to a user. The notification or alert may identify an action to be taken by the user, as described in further detail below.

The product of DS_{baseline} and N*SDev is referred to as a baseline threshold data signature. To determine if human intervention is required, current data signature 318, which has been weighted, is compared to baseline data signature 314 that has also been subject to the weighing factors of one of the health reference profiles at step 324, and multiplied by a multiple of the baseline data's standard deviation. If the data series representing the current data signature 318 is greater than the data series representing baseline data signature 314 multiplied by the predetermined multiple N of the baseline data's standard deviation, then a significant behavioral change in the animal has occurred between the time the baseline data was collected and the time that the current data was collected, and the animal may require medical attention from the animal owner or health care professional as a result to maintain the wellness of the animal.

Referring again to FIG. 5, user interface dashboard system 304 is configured to facilitate diagnosing the health status of at least one animal and is communicatively coupled to user application 308 through data analysis system 302. In the exemplary embodiment, user interface dashboard may be implemented in any of workstations 134, 136, 138, 146, and 148 illustrated in FIG. 2. User interface dashboard 304 provides continuous analytic service to any user with access to data analysis system 302. Specifically, user interface dashboard 304 may provide access to data analysis system 302 and its health determination 326 to an animal health care provider and enables the individual animal to be monitored remotely. Dashboard 304 further allows the health care provider to detect a health issue prior to the onset of clinical symptoms as well as remotely manage any chronic health conditions of the animal without having to physically examine the animal.

User interface dashboard 304 in the example shown includes an alert system 328, an action items database 330, and a message system 332. In the exemplary embodiment, alert system 328 generates an alert based on health assessment 326 and sends the alert via data analysis system 302 to user application 308. Specifically, based on the determined health assessment 326 or other determined changes between baseline signatures 314 or 322 and current signature 318 in view of reference health and physiological state profiles 324, alert system 328 sends an alert to at least one of the animal's owner/caretaker or the animal's health care provider. For example, if data analysis system 302 generates a health assessment 326 having a certain score that is outside a predetermined range indicating the animal to be healthy or indicating a significant change in animal behavior, then alert system 328 sends an alert to the at least one of the animal's owner/caretaker or the animal's health care provider notifying them that the animal may need further attention.

In an exemplary embodiment, user interface dashboard 304 also includes an action items database 330 that provides a user such as the animal's health care provider with a number of options to treat the animal Action items 330 displayed on dashboard 304 are based on the alert triggered by alert system 304. Alternatively, action items 330 displayed on user interface dashboard 304 may be independent of the alert triggered by alert system 304. The animal's health care provider chooses which action item 330 will best treat or prevent a condition reflected in the animal's health assessment 326 before the condition progresses further. Example action items 330 include but are not limited to: 1) alter the animal's diet to improve health or productivity of the animal; 2) adjust dosages of medications or other treatments to maximize effectiveness and minimize side effects; 3) flag the animal for closer monitoring; and 4) prepare the animal for reproduction if the alarm indicates an estrus or birthing event in the case of an animal breeder. A similar user interface dashboard could be presented to the animal owner or another person including recommendations to animals having a diagnosed condition or even for healthy animals. In the case where the health assessment reveals an animal to be in good health, tips and recommendations may be presented to a user such as the animal owner for possible consideration to improve, optimize or maintain certain healthy attributes of the animal over time.

User interface dashboard 304 may also include message system 332 that allows a user such as an animal health care provider to provide instructions or recommendations to data analysis system 302 that are then relayed to the animal's owner or caretaker's user application 308. If health assessment 326 indicates a serious medical issue, then message system 332 enables the health care provider to quickly contact the animal's owner or caretaker and provide instructions for treatment without physically examining the animal. User interface dashboard 304 provides the animal's health care provider with continuous updates to monitor changes in the animal's health status based on the action item 330 undertaken.

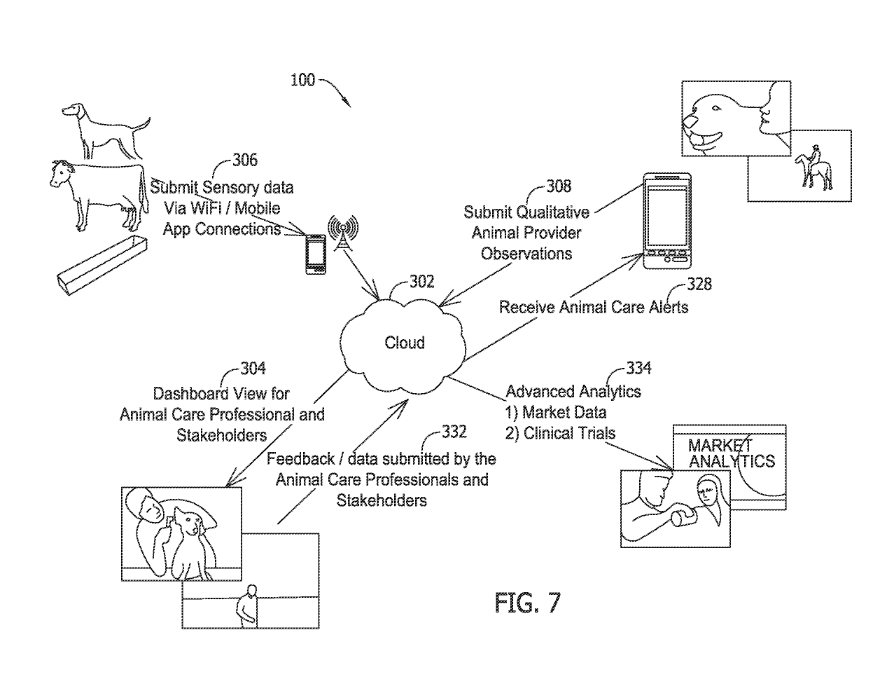

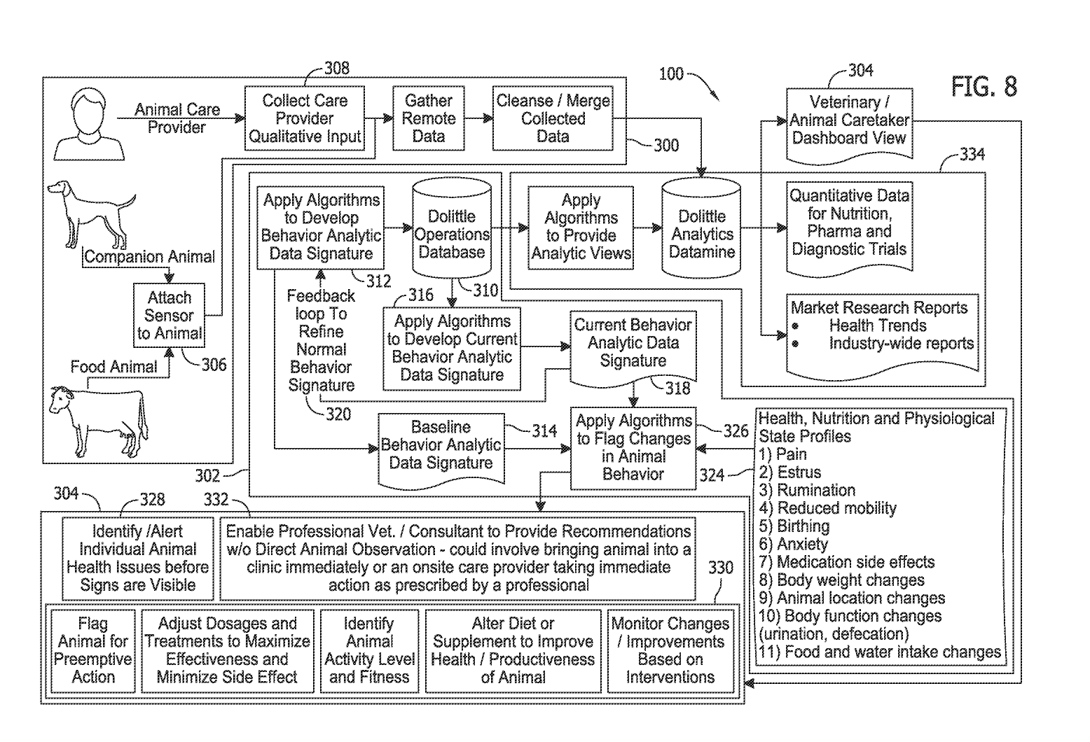

FIG. 7 is a detailed schematic diagram of the exemplary AHM system 100 shown in FIG. 5. FIG. 8 is a process flow diagram of animal health management system 100, as shown in FIG. 5, configured to monitor, manage, and diagnose the health status of at least one animal to predict whether or not an illness is oncoming before visible signs of the illness occur. AHM system 100 includes at least one of data collection system 300, data analysis system 302, and user interface dashboard system 304, as described in detail above. Furthermore, AHM system 100 may be used with companion animals such as dogs and cats and also with livestock animals such as cattle and pigs. Alternative embodiments of AHM system 100 may be used with both companion animals and livestock animals simultaneously. Additionally, AHM system 100 may be used with only a single animal, whether a companion or a livestock animal. Alternatively, AHM system 100 may be used with a population of animals such as multiple companion animals or a herd of livestock animals.

AHM system 100 also includes an advanced analytics system 334 that is configured to analyze data collected by data collection system 300 with the algorithms and relationships described above to determine individualized health assessments that may provide unique, quantitative insights on previously un-measured aspects of animal care and behavior patterns. Such analysis may be used during nutrition, pharmaceutical, and diagnostic trials to remotely monitor the health of at least one animal and its behavior or health changes due to implementing one or more treatments. For example, such treatments may include: providing an animal with a pharmaceutical drug, changing the diet or nutrition intake of the animal, or initiating a physical therapy rehabilitation program. Advanced analytics system 334 may also include generating industry-wide marketing research reports that provide an analysis of the results of the various treatments implemented during the animal's clinical trial.

By virtue of the system, methods and interfaces described, data analytics are possible that are not using conventional systems. For example, individualized collection of multiple data points corresponding to different health parameters for an individual animal over time may produce insights unique to that animal that may extend the life of the animal, as well as extend the use, enjoyment and nurturing of the animal by its owner. Such unique insights allow aspects to individualized treatment that heretofore have not been realized. Instead of generalized assumptions regarding “normal” or healthy conditions of animal, assessment of normal or health conditions are made using the animal's own baseline data, which may or may not correspond to traditional assumptions of what is or is not normal or healthy for a particular type of animal. From a research perspective, the processing of such data and the production of baseline signatures may prove invaluable. The baseline signatures of individual animals may be compared to other baseline signatures of animals of the same type and extrapolated to define trends and optimize operation of the system even further. In other words, as data is collected, the system may become progressively better at developing more accurate profiles and algorithms to assess individual animals, as well as populations of animals of certain types.

By virtue of the AHM system 100, even a relatively sick animal (as compared to healthy ones) can be provided an individualized baseline data signature, and changes in that baseline signature can provide meaningful insight into more effective treatments for whatever ails it. As such, instead of simply distinguishing animals having a certain condition from those that do not as existing animal monitoring systems do, the AHM system can assess the health of sick animals and reveal its health improvements or health deterioration over time. Because the AHM system 100 can monitor combinations of healthy animals and unhealthy animals at that same time but still in an individualized manner, much insight can be derived concerning the effectiveness of medical treatments for unhealthy animals, preventative health care considerations for healthy animals, and particular susceptibility of vulnerability of particular animals or types of animals to certain conditions.

From a feedlot management perspective, changes in baseline data signatures amongst a number of animals may provide a means by which a feedlot may be managed more efficiently as the effects of changes in the feedlot can be observed in the animals in more or less real time. Changes in baseline signatures may also reveal animal conditions that are expected but otherwise difficult to efficiently oversee in many instances. For example, a change in the baseline signature of certain animals may indicate a proper breeding cycle or that an animal birth event is imminent. Feedlot managers may accordingly more effectively direct resources to the places needed at the proper time when provided with such information.

The data collected by the AHM system 100 may be made available, via unique user interfaces, to parties other than those specifically mentioned above or for other purposes than those described thus far. For example, an animal breeder may be interested in the health care assessments, the data signatures, and profiles of individual animals, as well as collective data for certain breeds of animals in order to make decisions regarding reproduction. Pharmaceutical and/or vaccine manufacturers may be provided access, via a unique user interface, to establish norms for experimental animal drugs and dosages. Animal food manufacturers may be provided access, via a unique user interface, to animal data and data signatures to develop special formulations of food, and also to further optimize existing formulations. Veterinarians and animal health care providers may be provided access, via unique user interfaces, for data and information that is beneficial in treating an animal that is not being monitored by the system but is the same type as an animal that is being monitored by the system.

Still further, regional and local effects may be analyzed in a way that heretofore has not been possible. For example, data signatures can be collected for certain dog breeds in a particular suburb, and those data signatures can be compared to data signatures for the same dog breeds in different suburbs and against the same or other larger metropolitan areas. Continuing with this example, the data signatures of certain dog breeds in the Midwest of the United States may be compared with data signatures of the same dog breeds residing on the East Coast, West Coast or Southern United States. To the extent that certain conditions are more or less prevalent in certain geographic areas, different steps can be taken by animal owners and animal health care providers to avoid negative consequences. Likewise, animal data signatures can be compared for animals in different countries or even on different continents, and can be factored into the determination of normal or healthy baseline conditions for animals being monitored. In other words, the normal baseline signatures between two animals of the same type and breed may differ depending on their geographical location, and the inventive system described is uniquely situated to account for such differences. Signatures of different types of animals and different breeds of the same animal can be cross-compared to evaluate environmental influences and other factors in a holistic way.

In contemplated embodiments the baseline data signatures of animals being monitored may be dynamic and self-adjusting over time. For example, a determined data signature of a seven year old dog may be considered normal or healthy, while the same data signature would may not be considered normal or healthy for a two year old dog. Over the lifetime of the same dog, the baseline data signatures at various points in time may be expected to naturally change, and the system can intelligently account for this too. Various charts and graphs and other types of graphic information may be made available to users of all types to more readily understand the effects of age.

Various levels of health assessment may further be made available in various adaptations of the system. For example, a dog breeder may desire a higher bar for a normal or healthy data signature and/or a greater sensitivity to changes in the baseline than your typical dog owner. Likewise, a show dog may be more closely monitored than other dogs, and the system user(s) may accordingly select different modes of analysis. For example, dog breeders or show dog owners may be provided different versions of the AHM system 100 or otherwise dog breeders or show dog owners may be able to select which type of analysis is preferred. A drop down menu, for example, may be provided to compare an individual dog to breeder dogs, show dogs, or regular dogs of the same breed. Similar considerations may apply to racehorses, show horses, working horses, and horses primarily for recreational use. As another example, Angus beef ranchers may desire a different type of evaluation than non-Angus beef ranchers. Various other adaptations are possible.

In another aspect, AHM system data may be made available, via unique interfaces, to persons that are not current animal owners for educational purposes. As one example, a person interested in acquiring a dog may peruse the data as processed by the system to evaluate expected health care issues of dog breeds of various types. As noted above, the data may be tailored to specific geographic areas where the dog will reside and as such different users may receive different data.

The AHM system continuously updates its data and refines its algorithms over time for increased accuracy as more data is collected. Certain conditions and diagnoses may be made possible via the data collection and processing that were not heretofore possible to detect or diagnose.

The AHM system 100 may generate reports, individually and collectively, to comprehensively evaluate a variety of animals for further study and review. The level of information available may vary depending on user status. For example, an animal owner may be provided a first amount of informational feedback upon request and in contemplated embodiments an animal owner may primarily be provided with summary information in the form of charts and graphs and limited displays. A health care provider, however, may be provided, in addition to the summary information provided to the animal owner, supporting data for review by the animal health care provider. A scientist may be provided even more data than the animal care provider. Each user can identify the type of access desired in one of the display screens. In contemplated embodiments, the users of the system may subscribe, with the subscription being based on the level of data access desired, including free subscriptions, if desired, for certain types of users. Additionally, a user may select between novice and expert displays and feedback.

Beneficial embodiments of an AHM system have been disclosed for monitoring, managing, and diagnosing the health and/or behavior of at least one animal. In one embodiment, the system includes a data collection system, a data analysis system, and a user interface dashboard system. The data collection system facilitates monitoring the health of the at least one animal and includes a sensor device coupled to the at least one animal and including a plurality of sensors, wherein the sensor device is configured to collect and transmit sensed data relating to the at least one animal. The data collection system also includes a user application configured to receive and transmit observed data that is input to the user application by a user.

The data analysis system facilitates managing the health of the at least one animal and includes an operations database configured to receive the sensed data and observed data from the data collection system. A baseline data signature is generated by the data analysis system based on a first set of data from the operations database. A current data signature is generated by the data analysis system based on a second set of data from the operations database, wherein the first set of data is collected before the second set. The baseline data signature is continuously updated by a feedback loop to generate a revised baseline data signature that incorporates more recent data into the original baseline data signature. The data analysis system further includes a reference profile database comprising a plurality of health state profiles based at least on the sensed data collected from the at least one animal. The baseline data signature, said current data signature, and said reference profiles database are analyzed to determine a health assessment and/or to identify a behavior change profile of the at least one animal. The health assessment serves as a predictive indicator to predict whether or not an illness is oncoming before visible signs of the illness occur.

The user interface dashboard system facilitates diagnosing the health of the at least one animal and includes an alert system configured to transmit an alert to at least one of the animal's owner or the animal's health care provider if the determined health assessment is outside a predetermined range or exceeds a predetermined threshold. The user interface dashboard system further includes an action item database comprising a plurality of action items from which the at least one animal's health care provider can chose to treat the at least one animal. A message system within the user interface dashboard system is configured to facilitate a message being sent between the at least one animal's owner and the at least one animal's health care provider.

As will be appreciated based on the foregoing specification, the above-described embodiments of AHM system 100 may be implemented using computer programming or engineering techniques including computer software, firmware, hardware or any combination or subset thereof. Any such resulting program, having a non-transitional computer readable medium or computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

The systems and processes are not limited to the specific embodiments described herein. In addition, components of each system and each process can be practiced independent and separate from other components and processes described herein. Each component and process also can be used in combination with other components and processes.

The benefits and advantages of the inventive concepts are now believed to have been amply illustrated in relation to the exemplary embodiments disclosed.

An exemplary embodiment of method for remotely assessing a health state of at least one non-human animal has been disclosed. The method is implemented with at least one computing device including at least one processor in communication with at least one memory, the method comprising: generating a baseline data signature of at least one non-human animal using the at least one processor based on a first set of collected data; generating, by the at least one processor, a first current data signature of the at least one non-human animal based on a second set of collected data and at least one reference health state profile; multiplying the baseline data signature by a threshold factor to generate a baseline threshold data signature; comparing by the at least one processor, the baseline threshold data signature to the first current data signature; and generating a health assessment of the at least one non-human animal.

Optionally, generating the first current data signature may also include: collecting the first set of collected data from a plurality of sensors of different types at a plurality of predetermined time intervals over a first predetermined time period, wherein each one of the different types of sensors in the plurality of sensors of different types collects data related to a particular health parameter of the at least one non-human animal; collecting the second set of collected data from the plurality of sensors of different types at the plurality of predetermined time intervals over a second pre-determined time period; determining a plurality of behavioral event scores using the at least one processor based on the first and second collected data sets, wherein each of the plurality of behavioral event scores represents a sum of variances of a particular health parameter measured by a first type of sensor of the plurality of sensors of different types in the first and second collected data sets over corresponding predetermined time intervals; and applying a weighing factor to each of the plurality of behavioral event scores, wherein the weighing factor is based on the at least one reference health state profile, and wherein each behavioral event score is weighed based on the significance of the particular health parameter of the behavioral health score to the at least one reference health state profile.

As further options, at least one reference health state profile may include a predetermined health, behavior, or physiological state profile.

The method may also include generating a second current data signature using the at least one processor based on the second set of collected data and at least another reference health state profile that is different from the at least one health state profile. Multiplying the baseline data signature by the threshold factor may further include multiplying a multiple of a standard deviation of the first data set to the baseline data signature.

The at least one non-human animal may include a plurality of non-human animals, and the method may further include repeating, by the at least one processor, the steps above to generate a health state assessment of each of the plurality of non-human animals. The plurality of non-human animals may include animals of different types, with the method comprising repeating, by the at least one processor, the steps above to generate a health state assessment of each of the different types of animals in the plurality of animals. The method may include comparing, by the at least one processor, the health state assessments of the different types of animals against corresponding different types of animals in different geographic locations.

Generating the health assessment of the animal may include a predictive diagnosis of a health condition. The method may also include generating at least one alert relating to the predictive diagnosis so that medical intervention may occur before adverse physical symptoms are manifested in the non-human animal.

Generating a baseline data signature of at least one non-human animal using the at least one processor based on a first set of collected data may include dynamically generating a revised baseline data signature based upon at least one collected set of data subsequent to the first set of collected data.

Generating a health assessment of the at least one non-human animal may also include: when a variation between the baseline threshold data signature and the first current data signature exceeds a predetermined amount, indicating a significant change in health of the at least one non-human animal in at least one aspect; and when a variation between the baseline threshold data signature and the first current data signature exceeds is less than a predetermined amount, indicating the at least one non-human animal to be in good health.

An exemplary embodiment of a system for remotely monitoring the health state of at least one non-human animal has also been disclosed. The system includes at least one computing device including at least one processor in communication with at least one memory, the at least one processor programmed to: generate a baseline data signature of the at least one non-human animal based on a first set of collected data; generate a first current data signature of the at least one non-human animal based on a second set of collected data; multiply the baseline data signature by a threshold factor to generate a baseline threshold data signature for the at least one non-human animal; compare the baseline threshold data signature to the first current data signature; and generate a health assessment of the at least one non-human animal.

Optionally, the at least one processor is further programmed to receive observed data related to the health state of the at least one non-human animal, wherein the observed data is input by a human user. The at least one processor may further programmed to update the baseline data signature over time via a feedback loop. The baseline and current data signatures may be based on data collected from a plurality of sensors of different types.

The at least one non-human animal may include a plurality of non-human animals, and wherein the at least one processor is further programmed to generate a health state assessment of each of the plurality of non-human animals. The plurality of non-human animals may include animals of different types, and wherein the at least one processor-based device may be programmed to generate a health state assessment of each of the different types of animals in the plurality of animals. The at least one processor may further programmed to compare the health state assessments of the different types of animals against corresponding different types of animals in different geographic locations.

The at least one processor may be programmed to predictively diagnosis a health condition of the at least one non-human animal, may further be programmed to generate at least one alert relating to the predictive diagnosis so that medical intervention may occur before adverse physical symptoms are manifested in the at least one non-human animal. The at least one processor is programmed to dynamically generate a revised baseline data signature based upon at least one collected set of data subsequent to the first set of collected data.

The at least one processor may also be programmed to: when a variation between the baseline threshold data signature and the first current data signature exceeds a predetermined amount, indicate a significant change in health of the at least one non-human animal in at least one aspect; and when a variation between the baseline threshold data signature and the first current data signature exceeds is less than a predetermined amount, indicate the at least one non-human animal to be in good health.

An exemplary embodiment of a computer program embodied on a non-transitional computer readable medium for evaluating and assessing a health state of at least one non-human animal has also been disclosed. The program includes at least one code segment for instructing at least one computing device including at least one memory and at least one processor in communication with the memory to: generate a baseline data signature of the at least one non-human animal based on a first set of collected data; generate a first current data signature of the at least one non-human animal based on a second set of collected data; multiply the baseline data signature by a threshold factor to generate a baseline threshold data signature for the at least one non-human animal; compare the baseline threshold data signature to the first current data signature; and generate a health assessment of the at least one non-human animal.

Optionally, the computer program further includes at least one code segment for instructing the at least one processor to receive observed data related to the health state of the at least one non-human animal, wherein the observed data is input by a human user. At least one code segment may also be provided for instructing the at least one processor to update the baseline data signature over time via a feedback loop. The baseline and current data signatures may be based on data collected from a plurality of sensors of different types.

The at least one non-human animal comprises a plurality of non-human animals, and the computer program may include at least one code segment for instructing the least one processor-based device to generate a health state assessment of each of the plurality of non-human animals. The plurality of non-human animals may include animals of different types, and the computer program may include at least one code segment for instructing the at least one processor to generate a health state assessment of each of different type of animal in the plurality of animals. At least one code segment may also be provided for instructing the at least one processor to compare the health states of the different types of animals against corresponding different types of animals in different geographic locations.

The computer program may also include at least one code segment for instructing at least one processor to predictively diagnosis a health condition of the at least one non-human animal. At least one code segment may also be provided for instructing the at least one processor to generate at least one alert relating to the predictive diagnosis so that medical intervention may occur before adverse physical symptoms are manifested in the at least one non-human animal.

The computer program may also include at least one code segment for instructing the at least one processor to dynamically generate a revised baseline data signature based upon at least one collected set of data subsequent to the first set of collected data. At least one code segment may be also be provided for instructing the at least one processor to: when a variation between the baseline threshold data signature and the first current data signature exceeds a predetermined amount, indicate a significant change in health of the at least one non-human animal in at least one aspect; and when a variation between the baseline threshold data signature and the first current data signature exceeds is less than a predetermined amount, indicate the at least one non-human animal to be in good health.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for remotely assessing a health state of at least one non-human animal, said method being implemented with at least one computing device including at least one processor in communication with at least one memory, the method comprising:
generating a baseline data signature of at least one non-human animal using the at least one processor based on a first set of collected data;
generating, by the at least one processor, a first current data signature of the at least one non-human animal based on a second set of collected data and at least one reference health state profile;
multiplying the baseline data signature by a threshold factor to generate a baseline threshold data signature;
comparing by the at least one processor, the baseline threshold data signature to the first current data signature; and
generating a health assessment of the at least one non-human animal.

2. The method of claim 1, wherein generating the first current data signature further comprises:
collecting the first set of collected data from a plurality of sensors of different types at a plurality of predetermined time intervals over a first predetermined time period, wherein each one of the different types of sensors in the plurality of sensors of different types collects data related to a particular health parameter of the at least one non-human animal;
collecting the second set of collected data from the plurality of sensors of different types at the plurality of predetermined time intervals over a second pre-determined time period;
determining a plurality of behavioral event scores using the at least one processor based on the first and second collected data sets, wherein each of the plurality of behavioral event scores represents a sum of variances of a particular health parameter measured by a first type of sensor of the plurality of sensors of different types in the first and second collected data sets over corresponding predetermined time intervals; and
applying a weighing factor to each of the plurality of behavioral event scores, wherein the weighing factor is based on the at least one reference health state profile, and wherein each behavioral event score is weighed based on the significance of the particular health parameter of the behavioral health score to the at least one reference health state profile.

3. The method of claim 2, wherein at least one reference health state profile includes a predetermined health, behavior, or physiological state profile.

4. The method of claim 1 further comprising generating a second current data signature using the at least one processor based on the second set of collected data and at least another reference health state profile that is different from the at least one health state profile.

5. The method of claim 1, wherein multiplying the baseline data signature by the threshold factor further comprises multiplying a multiple of a standard deviation of the first data set to the baseline data signature.

6. The method of claim 1, wherein generating the health assessment of the animal includes a predictive diagnosis of a health condition

7. The method of claim 6, further comprising generating at least one alert relating to the predictive diagnosis so that medical intervention may occur before adverse physical symptoms are manifested in the non-human animal.

8. The method of claim 1, wherein generating a baseline data signature of at least one non-human animal using the at least one processor based on a first set of collected data comprising dynamically generating a revised baseline data signature based upon at least one collected set of data subsequent to the first set of collected data.

9. A system for remotely monitoring the health state of at least one non-human animal, said system including at least one computing device including at least one processor in communication with at least one memory, said at least one processor programmed to:
generate a baseline data signature of the at least one non-human animal based on a first set of collected data;
generate a first current data signature of the at least one non-human animal based on a second set of collected data;
multiply the baseline data signature by a threshold factor to generate a baseline threshold data signature for the at least one non-human animal;
compare the baseline threshold data signature to the first current data signature; and
generate a health assessment of the at least one non-human animal.

10. The system of claim 9, wherein the at least one processor is further programmed to receive observed data related to the health state of the at least one non-human animal, wherein the observed data is input by a human user.

11. The system of claim 9, wherein the baseline and current data signatures are based on data collected from a plurality of sensors of different types.

12. The system of claim 9, wherein the at least one non-human animal comprises a plurality of non-human animals, and wherein the at least one processor is further programmed to generate a health state assessment of each of the plurality of non-human animals.

13. The system of claim 9, wherein the at least one processor is programmed to predictively diagnosis a health condition of the at least one non-human animal.

14. The system of claim 9, wherein the at least one processor is programmed to dynamically generate a revised baseline data signature based upon at least one collected set of data subsequent to the first set of collected data.

15. The system of claim 9, wherein the at least one processor is programmed to:
when a variation between the baseline threshold data signature and the first current data signature exceeds a predetermined amount, indicate a significant change in health of the at least one non-human animal in at least one aspect; and
when a variation between the baseline threshold data signature and the first current data signature exceeds is less than a predetermined amount, indicate the at least one non-human animal to be in good health.

16. A computer program embodied on a non-transitional computer readable medium for evaluating and assessing a health state of at least one non-human animal, the program comprising at least one code segment for instructing at least one computing device including at least one memory and at least one processor in communication with the memory to:
generate a baseline data signature of the at least one non-human animal based on a first set of collected data;
generate a first current data signature of the at least one non-human animal based on a second set of collected data;
multiply the baseline data signature by a threshold factor to generate a baseline threshold data signature for the at least one non-human animal;
compare the baseline threshold data signature to the first current data signature; and
generate a health assessment of the at least one non-human animal.

17. The computer program of claim 16, wherein the baseline and current data signatures are based on data collected from a plurality of sensors of different types.

18. The computer program of claim 16, wherein the at least one non-human animal comprises a plurality of non-human animals of different types, and the computer program comprises at least one code segment for instructing the at least one processor to generate a health state assessment of each different type of animal in the plurality of animals.

19. The computer program of claim 16, wherein the computer program comprises at least one code segment for instructing at least one processor to predictively diagnosis a health condition of the at least one non-human animal.

20. The computer program of claim 16, wherein the computer program comprises at least one code segment for instructing the at least one processor to dynamically generate a revised baseline data signature based upon at least one collected set of data subsequent to the first set of collected data.
